(19) United States
(12) Patent Application Publication
(10) Pub. No.: US 2022/0233840 A1
(43) Pub. Date: Jul. 28, 2022

(54) MECHANICAL CIRCULATORY SUPPORT SYSTEMS AND METHODS

(71) Applicant: The Foundry, LLC, Menlo Park, CA (US)

(72) Inventors: Hanson S. Gifford, III, Woodside, CA (US); Ashley Seehusen, Mountain View, CA (US)

(21) Appl. No.: 17/596,532

(22) PCT Filed: Jun. 13, 2020
(86) PCT No.: PCT/US2020/037650

§ 371 Date: Dec. 13, 2021

Related U.S. Application Data
(60) Provisional application No. 62/861,985, filed on Jun. 14, 2019.

(51) Int. Cl. A61M 60/35; A61M 60/109; A61M 60/427; A61M 60/113; A61M 60/38; A61F 2/24; A61M 60/232; A61M 60/279; A61M 60/405

(52) U.S. Cl. CPC A61M 60/35; A61M 60/109; A61M 60/427; A61M 60/113; A61M 60/405; A61F 2/2427; A61M 60/232; A61M 60/279; A61M 60/38

(57) ABSTRACT

Mechanical circulatory support systems and methods are disclosed herein. In some examples, the present technology comprises a system for providing cardiac support to a patient where the system comprises a first elongated shaft configured to receive a delivery catheter therethrough, a second elongated shaft, and a pressure source coupled to the first and second elongated shafts. The first elongated shaft may have a distal end portion configured to be intravascularly positioned at a first cardiovascular location, and the second elongated shaft may have a distal end portion configured to be intravascularly positioned at a second cardiovascular location downstream of the first location. Pressure generated by the pressure source pulls blood from the first location proximally through the first shaft to the pressure source, then pushes the blood distally through the second shaft and into circulatory flow at the second cardiovascular location, thereby providing mechanical circulatory support to the patient.

### CROSS-REFERENCE TO RELATED APPLICATION(S)

The present application claims the benefit of priority to U.S. Provisional Application No. 62/861,985, filed Jun. 14, 2019, which is incorporated by reference herein in its entirety.

### TECHNICAL FIELD

The present technology relates to mechanical circulatory support systems and associated methods of use. In particular embodiments, the present technology relates to mechanical circulatory support systems for use in conjunction with catheter-based heart therapies.

### BACKGROUND

Over the past 15 years, transcatheter (or percutaneous) procedures to address cardiovascular diseases have become increasingly popular, especially in the last 5 years as long-term data for these devices has been published. Transcatheter Aortic Valve Replacement (TAVR), (also referred to as Transcatheter Aortic Valve Implantation (TAVI)), Transcatheter Mitral Valve Repair (TMVr), and Transcatheter Mitral Valve Replacement (TMVR) all access the heart percutaneously through the arteries or veins. Prior to the transcatheter approach, valve replacement or repairs were done through “open” procedures that required a sternotomy. The adverse events of a sternotomy were common and severe including prolonged recovery times, pain, infection, and other morbidity. [1][2] The transcatheter approach has made these procedures safer and easier to recover from and has made therapies accessible to patients who would otherwise be too sick to undergo an open surgery.

Aortic stenosis is the calcification and narrowing of the valve between the left ventricle and the aorta. The disease can be asymptomatic, but progression can lead to angina, syncope, or heart failure. [3] Aortic stenosis has a prevalence of 0.4% in the general population and affects about 1.3 million people in the United States. [4] Treatments include balloon aortic valvotomy (BAV), in which a balloon is placed across the aortic valve and inflated to fracture the calcification and restore mobility to the valve leaflets, surgical aortic valve replacement (SAVR), an open procedure in which a mechanical or bio-prosthetic valve is implanted, and the increasingly popular transcatheter aortic valve replacement (TAVR or TAVI), in which a prosthetic valve is implanted percutaneously through the femoral artery, transapically, or through direct aortic access. [5] Though TAVR was originally indicated for high-risk patients who were not likely to tolerate open surgery, further studies have shown that TAVR and SAVR have similar risk profiles, driving increasing popularity of the transcatheter approach. [6][7]

Mitral valve regurgitation is a condition in which the mitral valve does not close properly, causing abnormal leaking of blood backwards from the left ventricle, through the mitral valve, into the left atrium, when the left ventricle contracts. Most patients are asymptomatic until there is left ventricular (LV) enlargement and systolic dysfunction, pulmonary hypertension, or the onset of atrial fibrillation. Symptoms include fatigue and labored breathing. [8] Mitral valve regurgitation affects 1.7% of the United States adult population, approximately 3.9 million people. [9] Mitral valve repair (MVR) and mitral valve replacement (MVRx), both “open” procedures, have good results in young, low surgical risk patients but are associated with high mortality and morbidity in older, higher surgical risk patients. The “MitraClip”, a device for transcatheter mitral valve repair became commercially available in the United States in 2014, has been shown over a series of studies to be a viable treatment option for high risk surgical patients. [10][11][12]

Mitral valve stenosis is a condition that causes mechanical obstruction between the left atrium and left ventricle. It is most commonly a secondary condition to rheumatic heart disease that causes a narrowing of the valve due to immobile mitral valve leaflets, fibrosis, thickening, shortening, fusion, and calcification of the chordae tendineae. The narrowing creates a pressure gradient across the valve which causes the left atrium to work harder. Over time mitral valve stenosis can cause congestive heart failure, systematic arterial embolism, hemoptysis, pulmonary hypertension and death. [13][14] Treatment of mitral valve stenosis includes percutaneous mitral balloon valvotomy, in which a balloon is placed across the valve and inflated, valve repair, which can be open commissurotomy and include placement of an annuloplasty ring, valve replacement, which can be done in an open or closed procedure, or transcatheter mitral valve replacement (TMVR), which is percutaneous delivery of a prosthetic valve. TMVR is still investigational in the United States.

In addition, there are other therapies which involve catheterization of the heart, such as RF Ablation of the left atrium and pulmonary veins to prevent atrial fibrillation or other rhythm abnormalities, placement of occlusion devices in the left atrial appendage to prevent stroke in patients with atrial fibrillation, and other therapies.

Despite the benefits of transcatheter procedures, the patient population eligible for these procedures is generally still very high risk. While the procedures offer long-term benefit, they can cause temporary disruption and stress to the heart. Patients are more likely to become hemodynamically unstable, leading to cardiogenic shock, heart failure, and/or death. In particular, repair or replacement of the mitral valve places an extra strain on the left ventricle over a period of hours or days as the ventricle adjusts to ejecting a lower stroke volume against a higher pressure. This improves the long-term health of the patient by reducing or eliminating regurgitant flow but can cause a dangerous period of short-term stress. Currently, patients with low left ventricular ejection fractions are not considered safe candidates for transcatheter mitral valve repair or replacement due to this increased acute strain on the ventricle.

In the recovery and readjustment period, it would be advantageous to “unload” the heart or decrease the demand placed on the heart's pumping capacity, thus decreasing the heart's need for oxygen and nutrients. By shifting work to a short-term mechanical circulatory assist device, the unloaded heart is more likely to remain hemodynamically stable and allow for recovery and fewer adverse events. [15][16]

Mechanical assist devices draw blood from the arterial system, either from inside the heart (left atrium or left ventricle) or from just beyond the aortic valve (ascending aorta or descending aorta). Blood is pumped using centrifugal, screw, peristaltic, impeller, or roller pumps. Blood can be returned to the circulatory system in several different ways. If the device is intraluminal, blood may be returned a few centimeters downstream in the aorta via the same catheter with the in-line pump. Other devices draw the blood out of the body, through an extracorporeal pump, and return it to the arterial system through the femoral artery or another major peripheral artery of the body. Alternatively, blood can be drawn from the venous circulation, such as from the inferior vena cava or right atrium, and run through an oxygenator as well as a pump before being returned to the arterial system.

There are also intra-aortic balloon pumps (IABP) that use counter pulsation to reduce systolic pressure and increase diastolic pressure, thereby increasing cardiac output and forward blood flow. The IABP balloon is placed in the descending aorta and rapidly inflated and deflated using helium. During diastole, the balloon inflates, which increases blood flow to the body's tissues, including the coronary arteries and driving heart perfusion. During systole, the balloon deflates, lowering aortic pressure and decreasing the afterload on the heart.

Existing mechanical assist devices require multiple complex steps to gain the necessary access to the appropriate portions of the circulatory system, such as the creation of transseptal access to access the left atrium from the femoral vein. These steps typically involve the introduction of new devices. These steps add cost, take additional time and cause additional stress to the patient. Accordingly, improved systems and methods for providing mechanical circulatory support are needed.

### SUMMARY

The present technology relates to mechanical circulatory support systems and associated methods of use. In particular embodiments, the present technology relates to mechanical circulatory support systems for use in conjunction with catheter-based heart therapies. The subject technology is illustrated, for example, according to various aspects described below, including with reference to FIGS. 1-27. Various examples of aspects of the subject technology are described as numbered clauses (1, 2, 3, etc.) for convenience. These are provided as examples and do not limit the subject technology.
1. A system for providing cardiopulmonary support to patients undergoing transcatheter procedures, wherein the same catheter used for delivering the therapy is also used to withdraw blood from the cardiovascular system.
2. The system of Clause 1, wherein the catheter comprises a guide catheter having a steerable distal portion.
3. The system of Clause 2, wherein the guide catheter comprises one or more positioning features extending into a lumen of the guide catheter, the positioning features configured to position a delivery catheter within the guide catheter lumen.
4. The system of Clause 2 or Clause 3, wherein the guide catheter comprises one or more holes in a distal portion of the guide catheter, the holes configured to receive blood therethrough.
5. The system of any one of Clauses 2 to 4, wherein the distal portion of the guide catheter is at least partially coated with heparin or another anti-coagulant coating.
6. The system of any one of Clauses 2 to 5, wherein the guide catheter is at least partially coated with heparin or another anti-coagulant coating
7. The system of any one of Clauses 2 to 6, wherein the guide catheter comprises a distal end portion, a proximal end portion, and an intermediate portion therebetween, wherein the intermediate portion is configured to be positioned within an artery or a vein of a patient.
8. An adaptor for accessing a guiding catheter to allow its use as a drainage cannula.
9. A guiding catheter which also has features which allow it to be used as a drainage cannula.
10. A method of treatment which allows for cardiopulmonary support during or immediately after cardiovascular procedures without the need for new cannulation.
11. A method of treatment comprising:
positioning a catheter with a distal portion disposed within or adjacent a heart of a patient;
advancing a treatment device through the catheter and into the heart;
withdrawing blood from the heart through the catheter to an extracorporeal pump; and
returning blood from the pump to a blood vessel of the patient.
12. The method of Clause 11, wherein the treatment device comprises a prosthetic valve.
13. The method of Clause 11, wherein the treatment device comprises a valve repair device.
14. The method of Clause 11, wherein the positioning the catheter comprises positioning the catheter into the ascending aorta or the left ventricle.
15. The method of Clause 14, wherein the catheter is advanced through the descending aorta.
16. The method of Clause 11, wherein positioning the catheter comprises positioning the catheter into the left atrium.
17. The method of Clause 16, wherein the catheter is advanced through the inferior vena cava.
18. A method of treatment comprising:
positioning a catheter with a distal portion disposed within or adjacent a heart of a patient and an intermediate portion disposed within an artery or a vein of the patient;
advancing a treatment device through the catheter and into the heart;
withdrawing blood from the heart through the catheter to an extracorporeal pump; and returning blood from the pump to a blood vessel of the patient.
19. The method of Clause 18, wherein the positioning the catheter comprises positioning a distal end portion of the catheter into the ascending aorta or the left ventricle.
20. The method of Clause 18, wherein positioning the catheter comprises positioning a distal end portion of the catheter into the left atrium.
21. A system for providing cardiac support to a patient, the system comprising:
a first elongated shaft defining a first lumen extending therethrough, the first shaft having a proximal end portion and a distal end portion, wherein the distal end portion is configured to be intravascularly positioned at a first cardiovascular location, and wherein the lumen of the first shaft is configured to slidably receive a catheter housing an interventional element in a low-profile state;
a second elongated shaft defining a second lumen extending therethrough, the second shaft having a proximal end region and a distal end region, wherein the distal end region is configured to be intravascularly positioned at a second cardiovascular location within an artery of the patient; and
a pressure source configured to generate pressure within the first lumen and the second lumen, wherein the pressure source is configured to be coupled to the proximal end portion of the first shaft and the proximal end region of the second shaft, and wherein pressure generated by the pressure source pulls blood from the first location proximally through the first shaft to the pressure source, then pushes the blood distally through the second shaft and into circulatory flow at the second cardiovascular location, thereby providing mechanical circulatory support to the patient.
22. The system of Clause 21, wherein the pressure source is configured to generate the blood flow while the catheter is positioned within and/or extending distally from the distal end portion of the first shaft.
23. The system of Clause 21 or Clause 22, wherein the pressure source is configured to be extracorporeally positioned while generating pressure.
24. The system of any one of Clauses 21 to 23, wherein the pressure source is configured to generate negative pressure in the first shaft and positive pressure in the second shaft.
25. The system of any one of Clauses 21 to 23, further comprising an oxygenator configured to oxygenate the blood as it flows between the distal end portion of the first shaft and the distal end region of the second shaft.
26. The system of any one of Clauses 21 to 25, wherein the first cardiovascular location is within one of the left ventricle, the left atrium, or the ascending aorta.
27. The system of any one of Clauses 21 to 26, wherein the second cardiovascular location is within one of the ascending aorta, the aortic arch, the descending aorta, the subclavian artery, or the femoral artery.
28. The system of any one of Clauses 21 to 27, wherein the distal end portion of the first shaft comprises a steerable region configured to bend at an angle relative to a longitudinal axis of the first shaft.
29. The system of Clause 28, wherein the steerable region is a first steerable region and the distal end portion of the first shaft further comprises a second steerable region configured to bend at a second angle relative to the longitudinal axis of the first shaft.
30. The system of Clause 29, wherein the first angle is equal to the second angle.
31. The system of Clause 29, wherein the first angle is greater than the second angle.
32. The system of any one of Clauses 21 to 31, wherein the distal end portion of the first shaft comprises a plurality of openings extending through a sidewall of the first shaft.
33. The system of any one of Clauses 21 to 32, wherein a radial dimension of the distal end portion of the first shaft decreases in a distal direction.
34. The system of any one of Clauses 21 to 33, wherein the first shaft comprises a plurality of projections extending radially inward from an inner surface of the first shaft.
35. The system of Clause 34, wherein some or all of the projections comprise a curved surface that is convex toward the first lumen.
36. The system of Clause 34 or Clause 35, wherein the projections are evenly distributed around a circumference of the inner surface of the first shaft.
37. The system of Clause 34 or Clause 35, wherein the projections are asymmetrically distributed around a circumference of the inner surface of the first shaft.
38. The system of Clause 37, wherein the projections are configured to position the catheter against a portion of the inner surface of the first shaft.
39. The system of any one of Clauses 21 to 38, wherein the proximal end portion of the first shaft comprises an outflow channel configured to fluidly couple to the pressure source.
40. The system of Clause 39, wherein the outflow channel is disposed at an angle relative to a longitudinal axis of the first shaft.
41. The system of any one of Clauses 21 to 40, wherein the proximal end portion of the first shaft is flared in a proximal direction.
42. The system of any one of Clauses 21 to 41, wherein the proximal end portion of the first shaft comprises a valve.
43. The system of any one of Clauses 21 to 41, wherein the proximal end portion of the first shaft comprises a seal.
44. The system of Clause 42 or Clause 43, wherein the valve or seal is configured to limit air and/or fluid displacement through the valve or seal under negative and/or positive pressure.
45. The system of any one of Clauses 21 to 44, wherein an outer surface of the proximal end portion of the first shaft includes threads or a lip configured to engage with a connector.
46. The system of any one of Clauses 21 to 45, wherein the proximal end portion of the first shaft is configured to engage with a cap such that the proximal end portion of the first shaft comprises a closed lumen.
47. The system of any one of Clauses 21 to 46, wherein the distal end region of the second elongated shaft comprises at least one opening through a sidewall of the second elongated shaft.
48. The system of any one of Clauses 21 to 47, wherein the distal end region of the second elongated shaft comprises an atraumatic distal terminus.
49. The system of any one of Clauses 21 to 48, wherein the distal end region of the second elongated shaft comprises an open lumen.
50. The system of Clause 49, wherein the distal terminus of the second elongated shaft is beveled.
51. The system of any one of Clauses 21 to 50, further comprising a connector configured to fluidly couple the proximal end portion of the first shaft and the pressure source.
52. The system of Clause 51, wherein the connector comprises a coupler configured to detachably couple to (a) the proximal end portion of the first shaft and/or (b) the pressure source.
53. The system of Clause 51, wherein the connector comprises a coupler and tubing configured to detachably couple to the coupler.
54. The system of Clause 52 or Clause 53, wherein the coupler comprises a hollow shaft defining a lumen extending therethrough, wherein the shaft is configured to be received within the first lumen of the first shaft.
55. The system of Clause 54, wherein a radial dimension of the hollow shaft decreases in a distal direction.
56. The system of Clause 54 or Clause 55, wherein the hollow shaft is configured to be inserted through and hold open the valve or seal of the first shaft.
57. The system of any one of Clauses 52 to 56, wherein the coupler comprises an attachment portion configured to receive the proximal end portion of the first shaft.
58. The system of Clause 57, wherein the attachment portion comprises threads.
59. The system of Clause 57, wherein the attachment portion comprises a snap-fit mechanism.
60. The system of Clause 57, wherein the attachment portion comprises a locking screw configured to engage an outer surface of the proximal end portion of the first shaft.
61. The system of any one of Clauses 52 to 60, the coupler further comprising a valve positioned within the lumen of the hollow shaft.
62. The system of Clause 61, wherein the valve is generally conical.
63. The system of any one of Clauses 52 to 62, the coupler further comprising a seal.
64. The system of Clause 63, wherein the seal is an elastomeric o-ring.
65. The system of any one of Clauses 52 to 64, wherein the coupler comprises an outflow channel configured to fluidly couple to the pressure source.
66. The system of Clause 65, wherein the outflow channel is disposed at an angle relative to the shaft.
67. The system of Clause 65 or Clause 66, wherein an outer surface of the outflow channel is threaded or barbed.
68. The system of any one of Clauses 52 to 67, wherein the coupler comprises a flush port.
69. The system of any one of Clauses 21 to 68, further comprising a connector configured to fluidly couple the proximal end region of the second shaft and the pressure source.
70. The system of Clause 69, wherein pressure generated by the pressure source causes blood to flow from the first location into the distal end portion of the first shaft, then proximally through the first lumen and first connector to the pressure source, then distally from the pressure source through the second connector and the second lumen to the distal end region of the second shaft, then into the artery.
71. The system of Clause 69 or Clause 70, wherein pressure generated by the pressure source causes deoxygenated blood to flow from a third cardiovascular location into the first shaft, then proximally through the first lumen and first connector to the pressure source, then distally from the pressure source through the second connector and the second lumen to the distal end region of the second shaft, then into the artery.
72. The system of any one of Clauses 21 to 71, wherein the first location is a left atrium.
73. The system of any one of Clauses 21 to 71, wherein the first location is a left ventricle.
74. The system of any one of Clauses 21 to 71, wherein the first location is an aorta.
75. The system of any one of Clauses 21 to 74, wherein the distal end portion of the first shaft is configured to be positioned across a septum.
76. The system of any one of Clauses 21 to 75, wherein the interventional element comprises a prosthetic mitral valve.
77. The system of any one of Clauses 21 to 75, wherein the interventional element comprises a prosthetic aortic valve.
78. The system of any one of Clauses 21 to 75, wherein the interventional element comprises a heart valve repair device.
79. A system comprising:
a bypass device comprising a first end region with an inlet, a second end region with an outlet, and a fluid path extending therebetween, wherein the first end region is configured to be intravascularly delivered to and positioned at a first cardiovascular location, and wherein the second end region is configured to be intravascularly delivered to and positioned at a second cardiovascular location within an artery of the patient; and
a pressure source disposed along the fluid path between the inlet and the outlet,
wherein a portion of the bypass device between the pressure source and the inlet is configured to receive a catheter containing an interventional device, and wherein, when the pressure source is activated, the pressure source pulls blood from the first cardiovascular location into the inlet, through the fluid path, and ejects the blood from the outlet to the second cardiovascular location.
80. The system of Clause 78, wherein the pressure source is configured to aspirate blood from the first location and eject blood to the second location while the catheter is positioned within the bypass device.
81. The system of Clause 79 or Clause 80, wherein the pressure source is configured to aspirate blood from a third cardiovascular location comprising deoxygenated blood.
82. The system of any one of Clauses 79 to 81, wherein the third cardiovascular location is a right atrium of the patient.
83. The system of any one of Clauses 79 to 82, wherein the third cardiovascular location is an inferior vena cava of the patient.
84. The system of any one of Clause 79 to 83, further comprising an oxygenator configured to oxygenate the blood as it flows between the first end region and the second end region of the bypass device.
85. The system of any one of Clauses 79 to 84, wherein the pressure source is a pump.
86. The system of Clause 85, wherein the pump is a centrifugal pump, a peristaltic pump, a pulsatile pump, or a roller pump.
87. A method of providing mechanical circulatory support to a patient, the method comprising:
positioning a distal end portion of a first elongated shaft at a first cardiovascular location proximate a treatment site at or near the patient's heart, the first shaft defining a first lumen therethrough;
advancing a delivery catheter through the first lumen of the first shaft to the treatment site, wherein the delivery catheter contains an interventional device in a low-profile delivery state;
performing an interventional procedure at the treatment site with the interventional device; positioning a distal end region of a second elongated shaft at a second cardiovascular location within an artery of the patient, the second shaft defining a second lumen therethrough; and
generating pressure within the first and second lumens to pull blood from the first cardiovascular location through the first shaft and the second shaft and eject the blood from the distal end region of the second shaft at the second cardiovascular location.
88. The method of Clause 87, further comprising withdrawing the delivery catheter from the first shaft prior to generating the pressure in the first and second lumens.
89. The method of Clause 87, wherein the pressure is generated while the delivery catheter is at least partially positioned within the first shaft.
90. The method of Clause 87, further comprising withdrawing the delivery catheter from the first shaft after generating the pressure in the first and second lumens.
91. The method of Clause 87, wherein positioning the distal end portion of the first shaft at a treatment location comprises a retrograde transfemoral approach.
92. The method of Clause 87, wherein positioning the distal end portion of the first shaft at the treatment site comprises an antegrade transseptal approach.
93. The method of Clause 87, wherein positioning the distal end portion of the first shaft at the treatment site comprises a transaortic approach.
94. The method of Clause 87, wherein positioning the distal end portion of the first shaft at the treatment site comprises a transapical approach.
95. The method of Clause 87, wherein positioning the distal end portion of the first shaft at the treatment site comprises a trans-subclavian approach.
96. The method of Clause 87, wherein positioning the distal end portion of the first shaft at the treatment site comprises a transaxillary approach.
97. The method of any one of Clauses 87 to 96, wherein the first cardiovascular location is within one of the left ventricle, the left atrium, or the ascending aorta.
98. The method of any one of Clauses 87 to 96, wherein the second cardiovascular location is within one of the ascending aorta, the aortic arch, the descending aorta, the subclavian artery, or the femoral artery.
99. The method of any one of Clauses 87 to 98, wherein the treatment site is the same as the first cardiovascular location.
100. The method of any one of Clauses 87 to 99, wherein the interventional procedure is a transcatheter aortic valve replacement.
101. The method of any one of Clauses 87 to 99, wherein the interventional procedure is a transcatheter mitral valve replacement.
102. The method of any one of Clauses 87 to 99, wherein the interventional procedure is a transcatheter mitral valve repair.
103. The method of any of Clauses 87 to 102, further comprising oxygenating the blood via an oxygenator in-line with the fluid pathway.
104. A system for providing cardiac support to a patient, the system comprising:

an inlet catheter defining a first lumen extending therethrough, the inlet catheter having a proximal end portion and a distal end portion, wherein the distal end portion is configured to be intravascularly positioned at a first arterial location, and wherein the lumen of the inlet catheter is configured to slidably receive a delivery catheter housing a prosthetic heart valve in a low-profile state;
an outlet catheter defining a second lumen extending therethrough, the outlet catheter having a proximal end region and a distal end region, wherein the distal end region is configured to be intravascularly positioned at a second arterial location; and
a pump configured to be coupled to the proximal end portion of the inlet catheter and the proximal end region of the outlet catheter, and wherein pressure generated by the pump pulls blood from the first arterial location proximally through the inlet catheter to the pump, then pushes the blood distally through the outlet catheter and into circulatory flow at the second arterial location, thereby providing mechanical circulatory support to the patient.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 depicts a mechanical circulatory support system of the present technology configured for use in conjunction with a typical TMVR and/or TMVr procedure.

FIG. 2 depicts a mechanical circulatory support system of the present technology configured for use in conjunction with a typical TAVR procedure.

FIG. 3 depicts a mechanical circulatory support system of the present technology configured for use in conjunction with a typical TAVR procedure.

FIGS. 4A-4D each depict a distal end portion of a second elongated shaft configured to be positioned within an arterial blood vessel in accordance with the present technology.

FIG. 5 depicts a first elongated shaft and a delivery catheter in accordance with several embodiments of the present technology.

FIGS. 6A and 6B are axial and isometric views, respectively, of a first elongated shaft and a delivery catheter in accordance with several embodiments of the present technology.

FIGS. 7A and 7B are axial and isometric views, respectively, of a first elongated shaft and a delivery catheter in accordance with several embodiments of the present technology.

FIG. 8 depicts a first elongated shaft and a delivery catheter in accordance with several embodiments of the present technology.

FIG. 9 depicts a first elongated shaft in accordance with several embodiments of the present technology.

FIG. 10 is a cross-sectional view of a proximal end portion of a first elongated shaft and a delivery catheter in accordance with several embodiments of the present technology.

FIG. 11A is a cross-sectional view of a proximal end portion of a first elongated shaft and a delivery catheter in accordance with several embodiments of the present technology.

FIG. 11B is an axial view of the valve of FIG. 11A.

FIG. 12 is a cross-sectional view of a proximal end portion of a first elongated shaft in accordance with several embodiments of the present technology.

FIG. 13 is a cross-sectional view of a proximal end portion of a first elongated shaft and a delivery catheter in accordance with several embodiments of the present technology.

FIG. 14 is a cross-sectional view of a proximal end portion of a first elongated shaft and a delivery catheter in accordance with several embodiments of the present technology.

FIG. 15 is a cross-sectional view of a proximal end portion of a first elongated shaft in accordance with several embodiments of the present technology.

FIG. 16 is a cross-sectional view of a proximal end portion of a first elongated shaft and a cap in accordance with several embodiments of the present technology.

FIG. 17 depicts a proximal end portion of a first elongated shaft in accordance with several embodiments of the present technology.

FIG. 18 depicts a proximal end portion of a first elongated shaft in accordance with several embodiments of the present technology.

FIG. 19A is a cross-sectional view of a coupler in accordance with several embodiments of the present technology.

FIG. 19B is a cross-sectional view of the coupler of FIG. 19A attached to a first elongated shaft and tubing, each in accordance with several embodiments of the present technology.

FIG. 20 is a cross-sectional view of a coupler in accordance with several embodiments of the present technology.

FIG. 21 is a cross-sectional view of a distal attachment portion of a coupler in accordance with several embodiments of the present technology.

FIG. 22 is a cross-sectional view of a distal attachment portion of a coupler in accordance with several embodiments of the present technology.

FIG. 23 is a cross-sectional view of a distal attachment portion of a coupler in accordance with several embodiments of the present technology.

FIG. 24 is an isometric view of a distal end portion of a second elongated shaft in accordance with several embodiments of the present technology.

FIG. 25 is an isometric view of a distal end portion of a second elongated shaft in accordance with several embodiments of the present technology.

FIG. 26 is an isometric view of a distal end portion of a second elongated shaft in accordance with several embodiments of the present technology.

FIG. 27 is an isometric view of a distal end portion of a second elongated shaft in accordance with several embodiments of the present technology.

### DETAILED DESCRIPTION

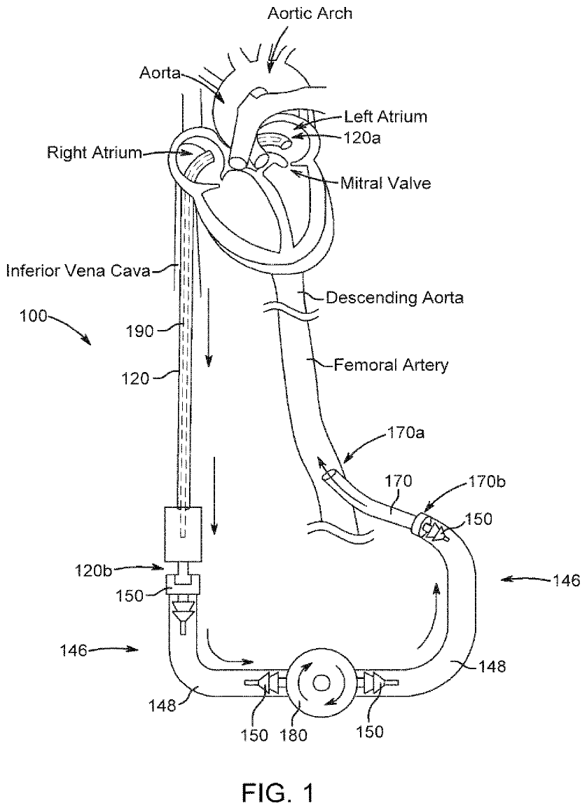

The present technology relates to systems and methods for providing mechanical circulatory support to patients undergoing or who have undergone catheter-based cardiovascular therapy. Some embodiments of the present technology, for example, are directed to providing mechanical circulatory support during or following transcatheter aortic valve replacement (TAVR) (also known as transcatheter aortic valve implantation (TAVI)), transcatheter aortic valve repair, transcatheter mitral valve replacement (TMVR), and/or native mitral valve repair (TMVr). The support systems of the present technology take advantage of existing access to the certain portions of the circulatory system established during a catheter-based procedure (such as any of the aforementioned heart valve therapies). Unless specifically stated otherwise, the terms “circulatory system” or “circulatory path,” “vascular” or “vascular system,” and “cardiovascular” or “cardiovascular system,” as used herein, refer to the blood vessels, the heart, or both. Likewise, “arterial” refers to any portion of the heart or blood vessels containing oxygenated blood. By obviating the complex steps required to introduce new devices to gain access to the appropriate portions of the circulatory system, the support systems and methods of the present technology save time and money and reduce patient stress and recovery time. Specific details of several embodiments of the technology are described below with reference to FIGS. 1-27.

### I. Support System Overview

FIG. 1 depicts a support system 100 (or “system 100”) configured in accordance with several embodiments of the present technology. In some embodiments, for example as shown in FIG. 1, the system 100 comprises a first elongated shaft 120, a second elongated shaft 170, and a pressure source 180 configured to be fluidly coupled to both the first and second elongated shafts 120, 170. The first elongated shaft 120 has a proximal end portion 120b configured to be coupled to the pressure source 180 and a distal end portion 120a configured to be positioned at a first location within the circulatory system. The second elongated shaft 170 has a proximal end portion 170b configured to be coupled to the pressure source 180 and a distal end portion 170a configured to be positioned at a second location, typically within the arterial system. When activated, the pressure generated by the pressure source 180 directs blood from the first location through the first and second shafts 120, 170 to the second location, thereby providing mechanical support to the heart.

In some embodiments, the proximal end portion 120b of the first shaft 120 connects directly to the pressure source 180. For example, the proximal end portion 120b of the first shaft 120 may comprise a coupling portion (not shown) integrally formed with the proximal end portion 120b of the first shaft 120. In some embodiments, for example as shown in FIG. 1, the first shaft 120 connects to the pressure source 180 via a connector 146. The connector 146 may comprise one or more couplers 150 configured to detachably couple to the proximal end portion 120b of the first shaft 120 and/or the pressure source 180. Additionally or alternatively, the connector 146 may comprise tubing 148 configured to detachably couple to the proximal end portion 120b of the first shaft 120, the pressure source 180, and/or one or more couplers 150 (should the system 100 include any couplers 150). In some embodiments, the proximal end portion 120b of the first elongated shaft 120 is directly coupled to the tubing 148. For example, the proximal end portion 120b may comprise an integral coupling portion that connects directly to the tubing 148 without additional couplers. Additional details regarding the connection between the first shaft 120 and the pressure source 180 are discussed below.

In some embodiments, the proximal end portion 170b of the second shaft 170 connects directly to the pressure source 180. For example, the proximal end portion 170b of the second shaft 170 may comprise a coupling portion (not shown) integrally formed with the proximal end portion 170b of the second shaft 170. In some embodiments, for example as shown in FIG. 1, the second shaft 170 connects to the pressure source 180 via a connector 146. The connector 146 may comprise one or more couplers 150 configured to detachably couple to the proximal end portion 170b of the second shaft 170 and/or the pressure source 180. Additionally or alternatively, the connector 146 may comprise tubing 148 configured to detachably couple to the proximal end portion 170b of the second shaft 170, the pressure source 180, and/or one or more couplers 150 (should the system 100 include any couplers 150). In some embodiments, the proximal end portion 170b of the first elongated shaft 170 is directly coupled to the tubing 148. For example, the proximal end portion 170b may comprise an integral coupling portion that connects directly to the tubing 148 without additional couplers. Additional details regarding the connection between the second shaft 170 and the pressure source 180 are discussed below.

The pressure source 180 may be a pump, such as a centrifugal pump, a screw pump, a peristaltic pump, an impeller pump, a roller pump, and others. When coupled to the first and second shafts 120, 170 and in use, the pressure source 180 may be extracorporeally positioned, or may be implanted within the patient. The pressure source 180 may be configured to generate a negative pressure (i.e., suction) within a lumen of the first shaft 120 to increase the pressure differential between the patient's physiologic blood pressure and the pressure within the lumen of the first shaft 120, thereby drawing more blood into the first shaft 120. The pressure source 180 may be configured to generate a positive pressure within a lumen of the second shaft 170. This positive pressure is typically higher than the arterial pressure of the patient, causing blood to flow out of the second shaft 170 into the patient's arterial system.

As previously mentioned, the system 100 is configured to provide mechanical circulatory support during or following a catheter-based heart therapy, such as TAVR, transcatheter aortic valve repair, TMVR, or TMVr, using some or all of the same delivery system components used to perform the heart therapy. The first shaft 120, for example, defines a lumen sized to slidably receive therethrough one or more delivery system components and/or treatment elements configured to treat or facilitate treatment of one or more structures of the heart. The delivery system may comprise a guidewire, a delivery catheter, an elongated push member, and/or other components. The treatment element may be advanced through the guide catheter in a low-profile delivery state, either housed within a delivery catheter or exposed. Non-limiting examples of treatment elements include a prosthetic mitral valve implant, a prosthetic aortic valve implant, a mitral valve repair device, an aortic valve repair device, a patent foramen ovale (PFO) closure device, a left atrial appendage (LAA) occlusion device, an atrial septal defect (ASD) closure device, an ablation catheter, a ventricular partitioning device, a myocardial anchoring system, and other interventional elements for catheter-based heart therapies. The pressure source 180 may be coupled to the first shaft 120 during the transcatheter heart therapy, or may be coupled to the first shaft 120 after the delivery catheter has been withdrawn from the first shaft 120.

The specific location within the circulatory system for placement of the distal end portion 120a of the first shaft 120 depends on the type of transcatheter procedure/heart structure being treated. For example, the distal end portion 120a of the first shaft 120 may be configured to be positioned at a first cardiovascular location (a) within the left atrium, (b) within the left ventricle, and/or (c) within the aorta at a location just downstream of the aortic valve. When the system 100 is used in conjunction with a TMVR and/or a TMVr procedure, for example, the distal end portion 120a of the first shaft 120 may be positioned in the left atrium or the left ventricle. When the system 100 is used in conjunction with a TAVR or transcatheter aortic valve repair procedure, the distal end portion 120a of the first shaft 120 may be positioned within the left ventricle and/or within the aorta at a location just downstream of the aortic valve (such as along the ascending aorta, aortic arch, or descending aorta).

Regardless of the procedure, the distal end portion 170a of the second shaft 170 is configured to be positioned at a second cardiovascular location within the arterial circulation. For example, the second cardiovascular location may be at or within the femoral artery, the subclavian artery, the descending aorta, the ascending aorta, or the aortic arch.

In some embodiments, blood can be drawn from the venous circulation, such as from the inferior vena cava or right atrium. In such embodiments, the system 100 may include an oxygenator, the blood pulled from the circulation can pass through the oxygenator as well as the pressure source 180 before being returned to the arterial system.

As previously mentioned, in some embodiments the system 100 is configured for use in conjunction with a TMVR and/or TMVr procedure. In such embodiments, the first shaft 120 may be a guide catheter sized to receive a delivery catheter containing an interventional device for replacing and/or repairing the mitral valve. As shown in FIG. 1, a distal end portion 120a of the first shaft 120 can be positioned in the left atrium and the distal end portion 170a of the second shaft 170 can be positioned in the femoral artery. The first shaft 120 may be delivered to the left atrium through the femoral vein, iliac vein, inferior vena cava and right atrium via an antegrade transseptal approach (as shown) or through the femoral artery, aorta, and left ventricle via a retrograde transfemoral approach. In some embodiments, the first shaft 120 is a guide catheter configured to be delivered to the left atrium and/or left ventricle via an antegrade transseptal approach. In such embodiments, when the system 100 is in use and providing circulatory support, the first shaft 120 extends distally from the pressure source 180 through a patient's inferior vena cava, into the right atrium of the patient's heart, and across the septum into the left atrium, as shown in FIG. 1. The second shaft 170 may be configured to be advanced into the femoral artery, iliac artery, or descending aorta such that the distal end region 170a of the second elongated shaft 170 is positioned within one of these vessels, or into the aortic arch.

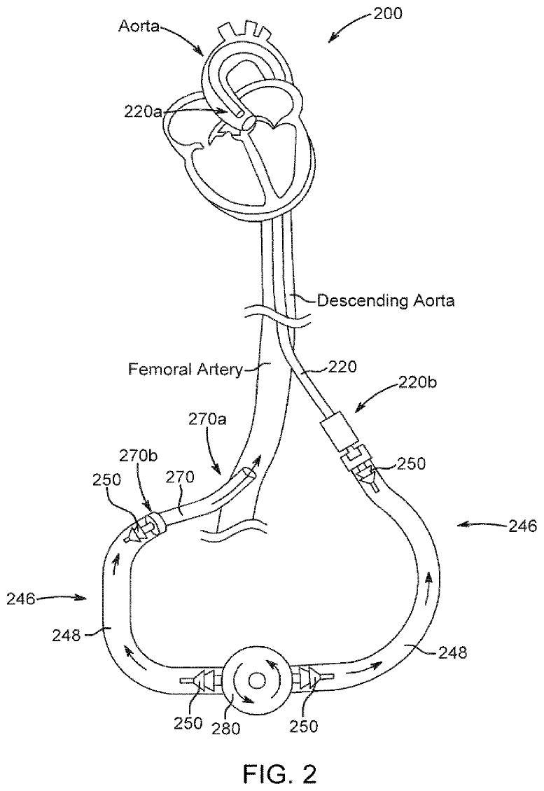

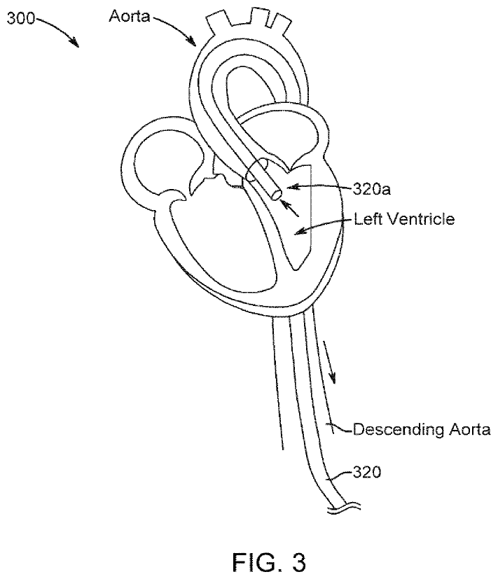

FIG. 2 depicts a system 200 of the present technology positioned within the cardiovascular system for use in conjunction with a TAVR procedure. The system 200 may comprise a first elongated shaft 220 having distal and proximal end portions 220a, 220b and a second elongated shaft 270 having distal and proximal end portions 270a, 270b. The proximal end portion 202b of the first elongated shaft 220 and/or the proximal end portion 270b of the second elongated shaft 270 may be configured to be attached to a pressure source 280. As shown in FIG. 2, at least when used in conjunction with a TAVR procedure, the distal end portion 220a of the first elongated shaft 220 may be positioned within the left ventricle or ascending aorta, and the distal end portion 270a of the second elongated shaft 270 can be positioned within the arterial system, such as within the femoral artery, iliac artery, or descending aorta. According to some embodiments, for example as shown in FIG. 3, the distal end portion 320a of the first elongated shaft 320 can be positioned within the left ventricle.

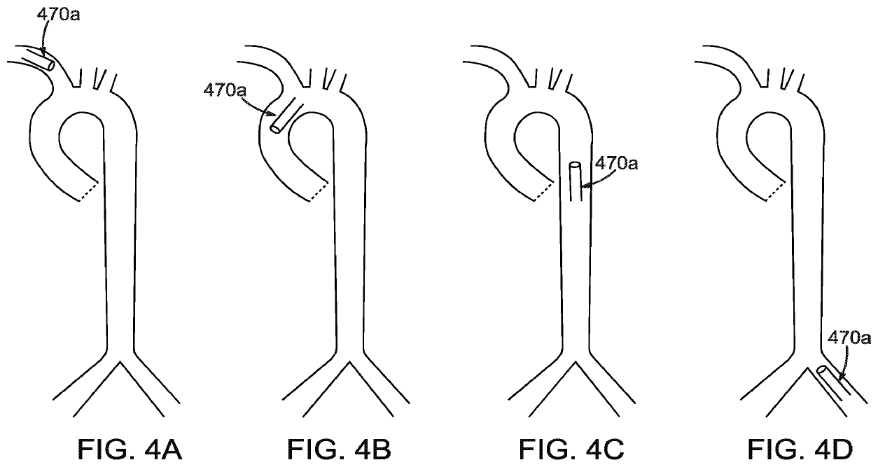

The distal end portion of the second shaft is typically positioned within a patient's arterial system. While FIGS. 1 and 2 depict the distal end portion of the second shaft positioned within the femoral artery, in some embodiments the distal end portion of the second shaft may be positioned elsewhere within the patient's cardiovascular system. For example, the distal end portion 470a of the second shaft may be configured to be positioned within a subclavian artery (FIG. 4A), an ascending aorta (FIG. 4B), a descending aorta (FIG. 4C), and/or a femoral artery (FIG. 4D).

### II. Selected First Shaft Embodiments

A first elongated shaft of the present technology may be formed of a polymeric and/or elastomeric material such as Pebax®, polyurethane, and other suitable materials. In some embodiments, an inner surface and/or an outer surface of the first shaft may include a coating configured to reduce or prevent clotting, damage to the vessel and/or heart wall, and/or an inflammatory response resulting from placement of the first shaft within a patient's cardiovascular system. Additionally or alternatively, the first shaft may include a reinforcement member, such as a coil, a braid, and others. In some embodiments, the reinforcement member is positioned within a sidewall of the first shaft, such as between the inner and outer surfaces.

According to some embodiments, the first shaft comprises at least one steerable region configured to bend along the longitudinal axis of the first shaft to reduce or prevent contact between the first shaft and the vessel walls as the first shaft is advanced through the vasculature. As such, the steerable regions herein reduce or prevent trauma to the vessel and/or formation of embolic debris, facilitate directing the shaft into the desired location, and facilitating delivery of an interventional device or other device to the appropriate location. The steerable region(s) may be controlled by a tensioning mechanism such as a longitudinal pull-wire positioned within the sidewall of the first shaft. Although longitudinal pull-wires are described herein, any suitable tensioning mechanism may be employed. The longitudinal pull-wire may be attached to the outer side of the inner surface and/or the inner side of the outer surface such that tensioning of the longitudinal pull-wire causes the first shaft to flex. In some embodiments, the first shaft comprises multiple steerable regions. For example, a first longitudinal pull-wire may be attached to the first shaft at a first location and a second longitudinal pull-wire may be attached to the first shaft at a second location proximal of the first location. The steerable regions may be configured to advantageously flex independently of one another. For example, when used in conjunction with a TAVR procedure, the first shaft may be positioned within the ascending aorta for delivery of a prosthetic aortic valve. To position the first shaft within the ascending aorta, the first (i.e., distal) steerable region may be flexed via tensioning of the pull-wire as the distal end portion of the first shaft is advanced from the descending aorta into the aortic arch and further into the ascending aorta. After the valve is delivered, the distal end portion of the first shaft may be advanced through the aortic valve into the left ventricle so that it can be used to withdraw blood from the left ventricle. During this advancement, the first steerable region may be flexed or straightened to minimize damage to the aortic valve and the second steerable region may be flexed while positioned within the aortic arch. The first shaft may comprise any number of suitable steerable regions.

A first elongated shaft of the present technology may have an outer diameter between about 14 and about 36 French, an inner diameter between about 12 and about 32 French, and/or a length between about 70 and about 160 cm. In some embodiments, the inner diameter of the first shaft may be greater than an outer diameter of a delivery catheter configured to be slidably received within the first shaft. For example, the first shaft may have a 30 French outer diameter and a 26 French inner diameter when configured for use with a delivery catheter having an outer diameter of 18 French. Oversizing of the first shaft relative to the delivery catheter may facilitate advancement of the delivery catheter through the first shaft to perform a therapeutic procedure. Additionally or alternatively, an oversized first shaft may permit blood to be withdrawn and/or mechanical circulatory support to be provided during the procedure by drawing blood through the annular space around the valve delivery catheter. Upon completion of the procedure, the oversized first shaft permits greater rates of blood flow through the first shaft as compared to a first shaft comprising a smaller inner diameter.

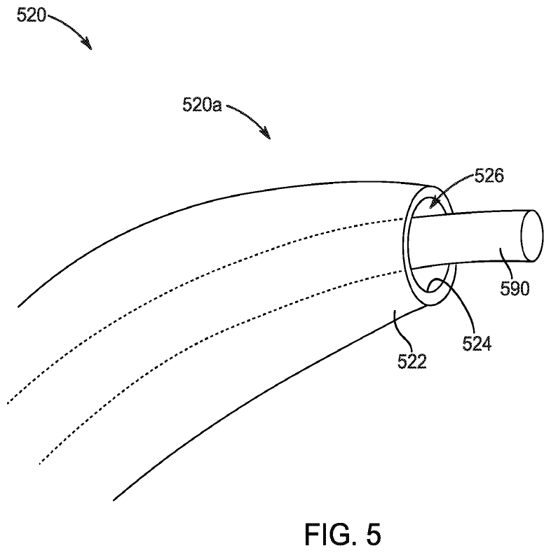

FIG. 5 is an isometric view of a distal end portion 520a of a first elongated shaft 520 and a delivery catheter 590 in accordance with several embodiments of the present technology. The first shaft 520 comprises an outer surface 522, an inner surface 524, and a lumen 526 defined by the inner surface 524. As shown, the delivery catheter 590 can be slidably received through the lumen 526 of the first shaft 520. In some embodiments, for example when the first shaft 520 is oversized relative to the delivery catheter 590, a radial dimension of the distal end portion 520a decreases in a distal direction. Distal tapering of the first shaft 520 can enable precise placement and orientation of the delivery catheter 590 during an interventional procedure.

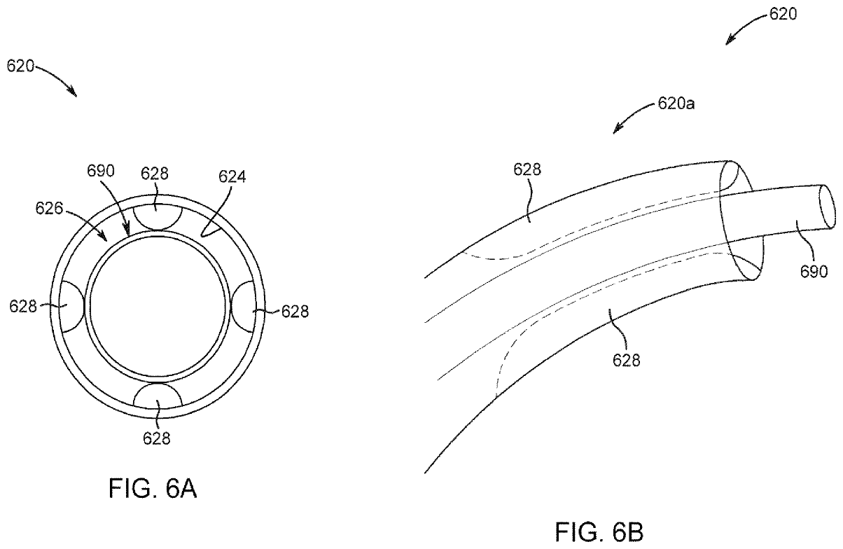

Additionally or alternatively, a first elongated shaft of the present technology may comprise internal features to facilitate positioning of the delivery catheter relative to one or both of the first shaft and the anatomy at the treatment site. For example, FIGS. 6A and 6B show axial and isometric views, respectively, of a first elongated shaft 620 having a plurality of guide members in accordance with several embodiments of the present technology. As shown, the guide members may comprise protrusions 628 extending inwardly towards the lumen 626 from the inner surface 624 of the shaft 620. The protrusions 628 may engage the outer surface of a delivery catheter (such as delivery catheter 690) while positioned in the lumen 626 to stabilize and guide advancement of the catheter 690. In some embodiments, the protrusions 628 are spaced apart about the circumference of the inner surface 624 such that, even when the delivery catheter 690 is positioned within the first shaft 620, blood can flow through the unobstructed regions of the lumen 626 between the protrusions 628.

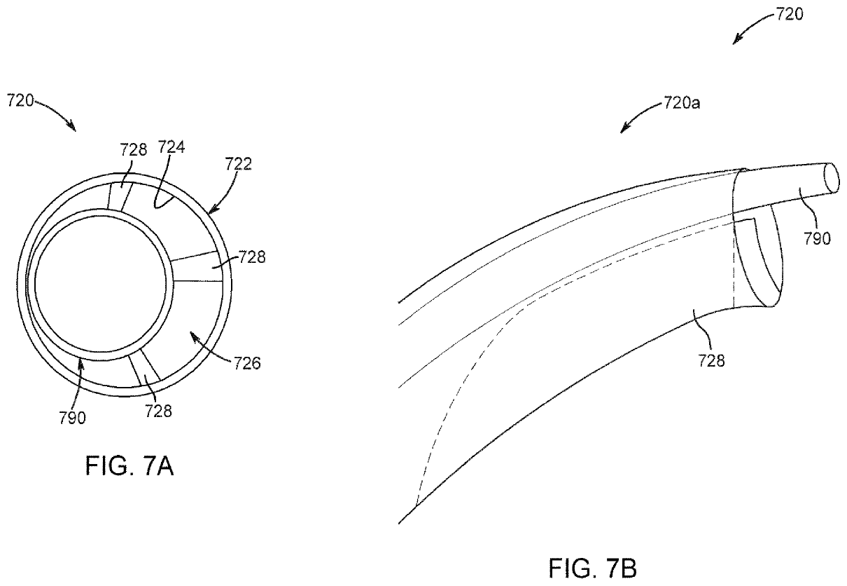

The protrusions 628 may be positioned along one or more discrete portions of the first shaft 620 or may extend continuously along the entire length of the first shaft 620. In some embodiments, the protrusions 628 are positioned along only a distal end portion 620a of the shaft 620 (as shown in FIG. 6A), only a proximal end portion, or only an intermediate portion. In some embodiments, the protrusions 628 are equally spaced around a circumference of the inner surface 624, for example to center the delivery catheter 690 within the lumen 626. According to several embodiments, the protrusions 628 may be asymmetrically arranged (for example as discussed below with reference to FIGS. 7A and 7B).

The protrusions 628 may be separate components coupled to the shaft 620 or may be unitarily formed with the shaft 620 (for example, via an extrusion process). The protrusions 628 may have any suitable cross-sectional shape, such as a hemispherical or rounded shape (see FIG. 6A), a square, a rectangle, a quadrilateral, a trapezoid (see FIG. 7A), a polygon, or any other suitable shape. All of the protrusions of a given shaft 620 may have the same shape and/or size, or some or all of the protrusions may have a different shape and/or size. In some embodiments, one, some, or all of the protrusions 628 comprise a rounded surface that is convex towards the lumen 626 and free of corners, for example as shown in FIG. 6A. Rounded protrusions may be advantageous for minimizing thrombus formation, whereas quadrilateral protrusions may be advantageous for stabilizing the delivery catheter and/or for ease of manufacture.

While FIG. 6A shows a first shaft 620 with four protrusions 628, in some embodiments the first shaft 620 may include more or fewer protrusions. For example, the first shaft 620 may include 1-20 protrusions, 2-18 protrusions, 3-15 protrusions, 3-8 protrusions, 4-10 protrusions, or other suitable numbers of protrusions.

FIGS. 7A and 7B are axial and isometric views, respectively, of a delivery catheter 790 positioned within a first elongated shaft 720 having an outer surface 722, an inner surface 724, and a lumen 726 defined by the inner surface 724. The first shaft 720 comprises guide members in the form of protrusions 728 extending radially inward from the inner surface 724. Here, the protrusions 728 are positioned asymmetrically around only a portion of a circumference of the inner surface 724 to position the delivery catheter 790 against or near the other portion of the inner surface 724 without protrusions 728 (see FIG. 7A). The semilunar space between the delivery catheter 790 and the opposing portion of the inner surface 724 may permit greater blood flow through the first shaft 720, as compared to the symmetrical annular space between the delivery catheter 690 and the inner surface 624 of the first shaft 620 in FIG. 6A. In some embodiments, the protrusions 728 may be positioned on the inner curvature of the distal end portion 720a of the first shaft 720 when it is flexed, so the delivery catheter 790 follows the outer curvature of the first shaft 720. In some embodiments, a tapered-tip dilator used to introduce the first shaft 720 over a guidewire may have corresponding slots to accommodate the protrusions 728.

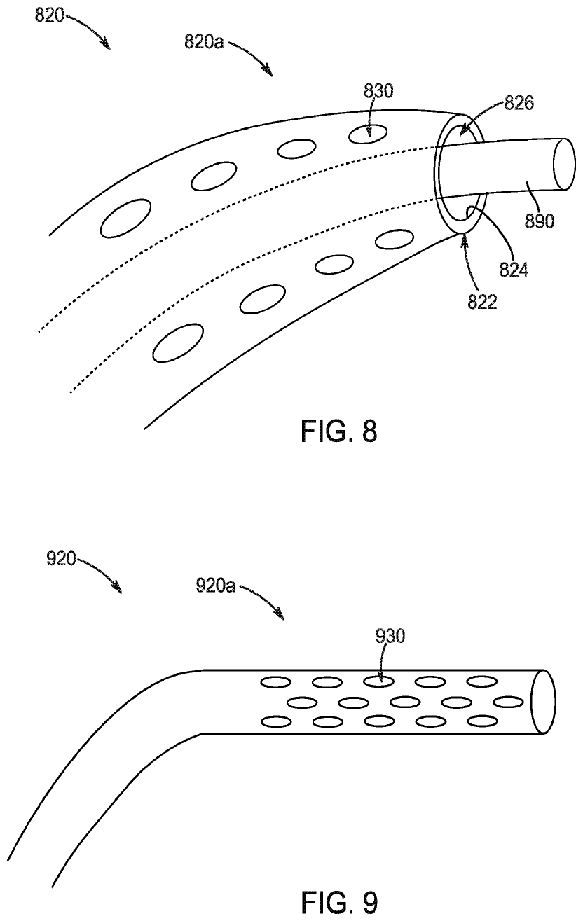

FIG. 8 shows a distal end portion 820a of a first elongated shaft 820 comprising an outer surface 822, an inner surface 824, and a sidewall extending therebetween. The first shaft 820 is shown in FIG. 8 with a delivery catheter 890 positioned in its lumen 826. In some embodiments, for example as shown in FIG. 8, the first shaft 820 can comprise one or more openings 830 extending through the sidewall to permit blood flow into the first shaft 820. The openings 830 may allow a sufficient volume of blood to flow into the first shaft to compensate for the tapered distal end portion of the delivery catheter 890. In some embodiments, the openings 830 are located at the distalmost 1-3 centimeters of the first shaft 820. The positioning of the openings 830 can be based at least in part on the intended blood withdrawal location (e.g., left atrium, left ventricle, aorta, etc.).

The number and locations of openings through a sidewall of the first shaft may be based at least in part on the geometry of the first shaft and/or the intended positioning of the first elongated shaft within the patient's heart and/or vasculature. For example, FIG. 9 depicts a distal end portion 920b of a first shaft 920 comprising a plurality of openings 930 around the circumference of the first shaft 920. The first shaft 920 of FIG. 9 comprises a greater number of openings 930 than the first shaft 820 of FIG. 8 and may thereby be configured for increased blood flow through the first shaft 920. The first shaft 920 may comprise any suitable number of openings. The openings 930 may be placed along a length of the first shaft 920 and/or about a circumference of the first shaft 920 to withdraw blood from a specific location within the patient's heart and/or vasculature. For example, if the distal end portion 920b of the first shaft 920 is intended to be placed in the left atrium via the inferior vena cava and right atrium (e.g., as depicted in FIG. 1), the portions of the first shaft 920 positioned within the right atrium and/or inferior vena cava may include openings 930. Such openings 930 allow more blood to be withdrawn through the first shaft 920, the blood withdrawn from the right atrium and inferior vena cava is deoxygenated. The system may comprise an oxygenator to add oxygen to such deoxygenated blood prior to reintroducing the blood into the patient's arterial system. The openings 930 may be sufficiently small to prevent kinking of the first shaft 920 or inadvertent passage of a delivery catheter through an opening 930.

To prevent blood leakage from the first shaft and/or air leakage into the first shaft, in some embodiments the proximal end portion of the first shaft comprises an adapter, a handle, and/or a housing to allow advancement, retraction, and/or torqueing of the first shaft. The proximal end portion of the first shaft may also comprise controls of any steerable region(s) of the first shaft. In some embodiments, the proximal end portion of the first shaft is configured to be attached to a rack along with a delivery catheter to stabilize the system components for precise delivery of an interventional element.

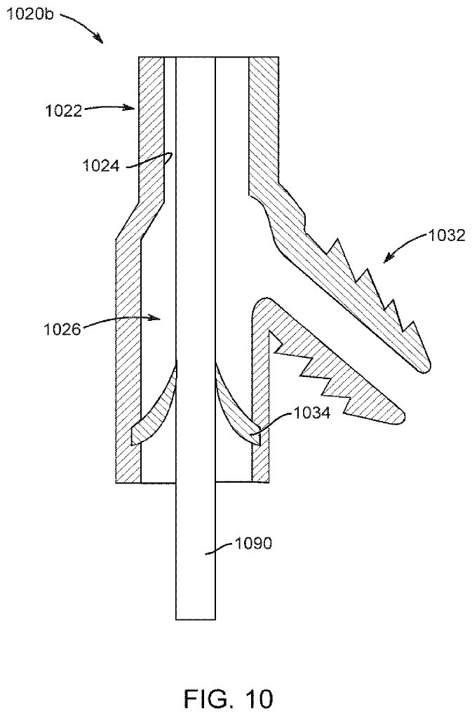

FIG. 10 is a cross-sectional view of a proximal end portion 1020b of a first elongated shaft 1020 of the present technology. The proximal end portion 1020b may also have the aforementioned handle and steering controls, not shown here. The first shaft 1020 comprises an outer surface 1022, an inner surface 1024, and a lumen 1026 defined by the inner surface. The proximal end portion 1020b of the first shaft 1020 may comprise an outflow channel 1032 disposed at an angle relative to a longitudinal axis of the first shaft 1020. The outflow channel 1032 may be configured to be coupled to a pressure source and/or a second elongated shaft of the present technology. In some embodiments, the angle between the outflow channel 1032 and the longitudinal axis of the first shaft 1020 is between about 30 degrees and about 330 degrees. In some embodiments, the angle between the outflow channel 1032 and the longitudinal axis of the first shaft 1020 is about 90 degrees, for example to minimize the length of the first shaft 1020.

The outflow channel 1032 may be configured to be coupled to tubing which is in turn coupled to the pressure source and/or the second shaft. In some embodiments, the outer surface 1022 of the outflow channel may be barbed, threaded, or otherwise configured to interlock with the pressure source, the tubing, and/or the second shaft. The outflow channel 1032 may be integrally formed with the first shaft 1020 (see FIG. 10), detachably coupled to the first shaft 1020, and/or rotatably coupled to the first shaft 1020. If rotatably coupled, the connection of the outflow channel 1032 to the first shaft 1020 may include o-rings or other seals to prevent leakage of air or fluid. In some embodiments, the proximal end portion 1020b of the first shaft 1020 comprises a port (not pictured) configured to remove air and/or blood from the lumen 1026 of the first shaft 1020 and/or introduce saline, radiopaque contrast dye, anticoagulants, medications, etc. into the lumen 1026 of the first shaft 1020.

As previously mentioned, a proximal end portion 1020b of the first shaft 1020 may be configured to receive a delivery catheter therethrough. To prevent blood leakage from the first shaft 1020 and/or the introduction of air into the blood stream, the proximal end portion 1020b of the first shaft 1020 may comprise a valve 1034 that receives and/or conforms to the delivery catheter 1090.

A proximal end portion of the first elongated shaft of the present technology may comprise a hemostatic valve or seal 1034 to prevent blood from advancing proximally beyond the valve or seal. The valves and seals described herein may be formed of any suitable material including synthetic rubbers or thermoplastics. In some embodiments, a single valve or seal may provide sufficient leakage protection. However, in some embodiments a reinforced or adjustable valve or seal and/or multiple valves or seals may be advantageous for providing leakage protection while mechanical circulatory support is being performed and pressure is being generated within the first shaft. The multiple valves or seals might be oriented in different directions, so that one prevents egress of air or fluid, and another prevents ingress of air or fluid. Valves and seals such as those described herein may also be employed in a second elongated shaft of the present technology.

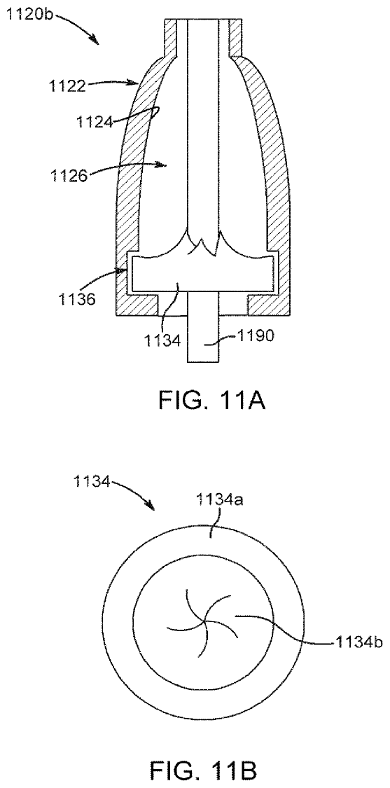

In some embodiments, for example as shown in FIGS. 11A and 11B, the proximal end portion 1120b of the first shaft 1120 may comprise a valve 1134 having an annular portion 1134a and a plurality of flaps 1134b. The annular portion 1134a of the valve 1134 may be received within a recess 1136 in the sidewall of the proximal end portion 1120b of the first shaft 1120. The flaps 1134b may be separated by slits such that an individual flap is movable relative to the other flaps towards the center of the valve 1134. Accordingly, the flaps 1134b may be configured to generally conform to a delivery catheter 1190 inserted through the valve 1134 and prevent blood or air passage through the valve 1134.

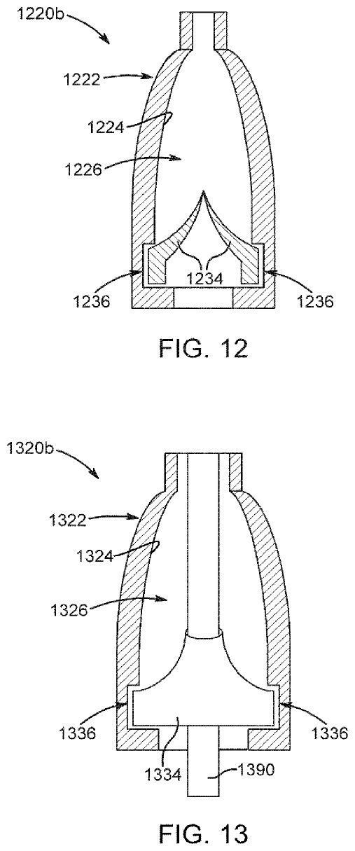

FIG. 12 depicts a proximal end portion 1220b of a first elongated shaft 1220 in accordance with several aspects of the present technology. As shown in FIG. 12, in some embodiments, the proximal end portion 1220b comprises a duckbill-style valve 1234 having two or more flaps received within a recess 1236 in the sidewall of the proximal end portion 1220b of the first shaft 1220. The tips of the flaps may be positioned in contact to prevent blood flow from the first shaft 1220 from progressing proximally beyond the valve 1234. This valve may be advantageous for preventing blood leakage during mechanical circulatory support initiated after an interventional procedure has been completed and a delivery catheter has been removed from the first shaft. Additionally or alternatively, the tips of the flaps may conform to a delivery catheter positioned within the first shaft (as shown in FIG. 10) to prevent blood leakage during mechanical circulatory support provided while the delivery catheter is positioned within the first shaft. The valve 1234 might also comprise a second valve oriented in the opposite direction. For example, the proximal end portion 1220b of the first shaft 1220 may comprise a second valve 1234 oriented in the opposite direction from the valve 1234 shown in FIG. 10 to prevent air and/or fluid ingress into the first shaft 1220.

In some embodiments, for example as shown in FIG. 13, a proximal end portion 1320b of a first shaft 1320 can comprise a valve 1334 configured to prevent blood leakage during mechanical circulatory support provided during an interventional procedure and/or while a delivery catheter 1390 is positioned within the first shaft 1320. The valve 1334 depicted in FIG. 13 comprises a generally continuous ring with a generally circular opening positioned at the center of the ring. The delivery catheter 1390 may be received through the opening in the valve 1334. The valve 1334 may have a generally conical shape, as shown in FIG. 13.

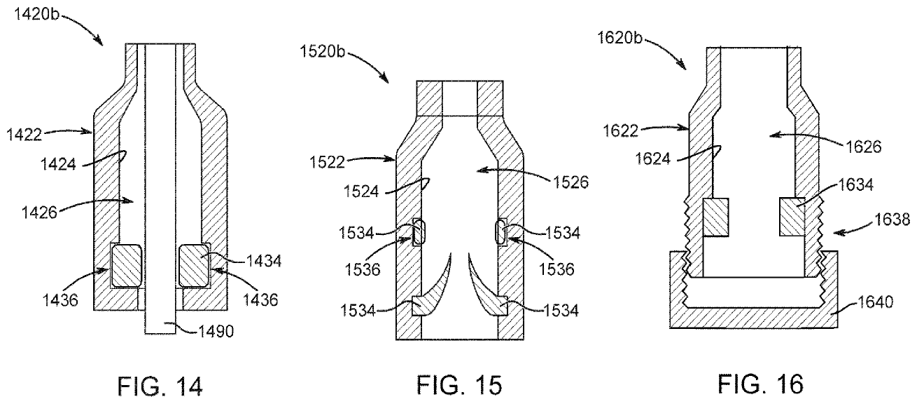

FIG. 14 is a cross-sectional view of a proximal end portion 1420b of a first elongated shaft 1420 including an o-ring seal 1434 received within a recess 1436 within a sidewall of the first shaft 1420. A delivery catheter 1490 may be received through the opening of the seal 1434 such that blood flow proximal of the seal 1434 is prevented while the delivery catheter 1490 is positioned within the first shaft 1420.

FIG. 15 shows a cross-sectional view of a proximal end portion 1520b of a first shaft 1520 comprising a two-stage valve 1534 configured to prevent blood leakage and/or air inflow during mechanical circulatory support. This may be most advantageous when mechanical circulatory support is provided while an interventional procedure is being performed and a delivery catheter 1490 is positioned within the first shaft 1520. As shown in FIG. 15, the two-stage valve 1534 may comprise an o-ring seal positioned distal of a duckbill-style valve. Any combination of suitable valves or seals, such those described elsewhere herein, may be positioned in series to form a two-stage valve.

A lumen of a proximal end portion of a first shaft may be completely closed when mechanical circulatory support is initiated after a delivery catheter has been removed from the first shaft. For example, as shown in FIG. 16, a proximal end portion 1620b of the first shaft 1600 may comprise an attachment portion 1638 configured to attach a cap 1640 to the first shaft 1600 and thereby close the lumen 1656 of the first shaft 1600. The attachment portion 1638 may comprise threads, barbs, or any other suitable attachment mechanism. In some embodiments, a cylindrical member may be positioned within the lumen 1626 of the proximal end portion 1620b and/or inserted through the valve(s) after the delivery catheter has been removed to prevent air or fluid leakage through the valve(s).

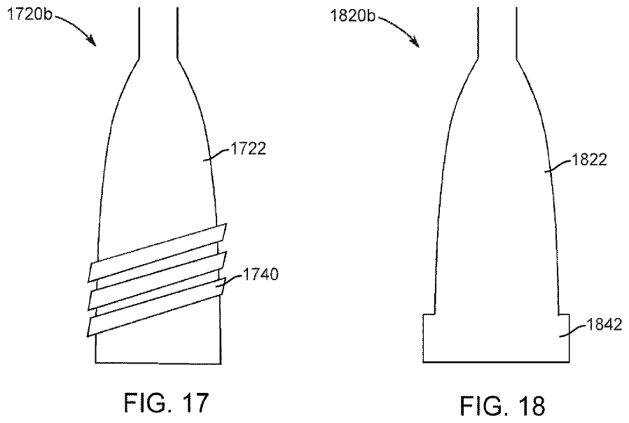

According to some embodiments, a proximal end portion of a first elongated shaft of the present technology may be configured to be attached to a connector. FIG. 17 depicts a proximal end portion 1720b of a first elongated shaft 1720 configured to attach to a connector in accordance with several embodiments of the present technology. The outer surface 1722 of the proximal end portion 1720b comprises threads 1740 configured to engage with female threads of a connector. The first shaft 1720 can comprise any suitable mechanism for attaching to a connector. For example, the proximal end portion 1820b depicted in FIG. 18 comprises a lip 1842 configured to engage with a corresponding attachment portion of a connector.

### III. Selected Coupler Embodiments

According to some embodiments, a system of the present technology may comprise a connector configured to attach a proximal end portion of a first shaft to a pressure source. The connector may comprise a tube and/or a coupler. In some embodiments, the tube is formed integrally with the coupler. The tube and coupler may be detachably coupled. In some embodiments, the first shaft is connected directly to the pressure source, to just a tube, or to just a coupler. The connector may be attached to the proximal end portion of the first shaft before, during, and/or after an interventional procedure (e.g., TAVR, TMVR, etc.). In some embodiments, the pressure source may be integral with the coupler.

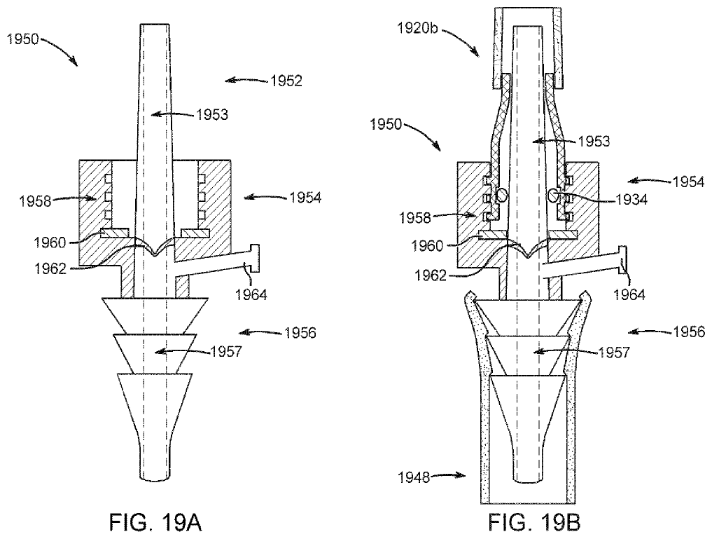

FIGS. 19A and 19B are cross-sectional views of a coupler 1950 in accordance with several embodiments of the present technology and a coupler 1950 attached to a proximal end portion 1920b of a first elongated shaft 1920 and a tube 1948, respectively. As shown in FIG. 19A, the coupler 1950 may comprise a shaft 1952 configured to be received within a lumen 1926 of the first shaft 1920 and/or to penetrate a valve and/or seal 1934 within the lumen 1926 of the first shaft 1920. Accordingly, the shaft 1952 may have a radial dimension that decreases in a distal direction (i.e., distally tapers) to penetrate the seal 1934 and hold the seal 1934 in an open position. In some embodiments, the shaft 1952 has a constant diameter to maximize the diameter of the lumen 1953. In some embodiments, the coupler 1950 may comprise a removable obturator configured to facilitate penetration seal 1934 of the first shaft by the coupler 1950 without damaging the seal 1934.

A lumen 1953 extending through the shaft 1952 is configured to permit blood to flow proximally from the first shaft 1920 through the lumen 1953 of the shaft 1952 of the coupler 1950. The coupler 1950 may comprise a one-way valve 1962 within the lumen 1953 of the shaft 1952 to prevent blood from flowing in the other direction through the lumen 1953 when mechanical circulatory support is not being supplied (i.e., no pressure is being generated in the lumen of the first shaft). In some embodiments, the coupler 1950 includes a port 1964 for withdrawing blood and/or air from the lumen 1953. It may be advantageous to maximize the diameter of the lumen 1953 to maximize blood flow during mechanical circulatory support. The wall thickness of the shaft 1952 may be minimized to maximize the diameter of the lumen 1953. The shaft 1952 may be formed of a polymer, metal, or another suitable material. However, forming the shaft 1952 of metal may facilitate minimizing the wall thickness of the shaft 1952.

The coupler 1950 comprises an outflow channel 1956 extending proximally from the shaft 1952 and having a lumen 1957 extending through the outflow channel 1956. The distal end portion of the lumen 1957 of the outflow channel 1956 is open to the lumen 1953 of the shaft 1952 and the proximal end portion of the lumen 1957 of the outflow channel 1956 is open to a lumen 1949 of the tube 1948 leading to the pressure source. An outer surface of the outflow channel 1956 may comprise a mechanism for attaching to the tube 1948. For example, the outflow channel 1956 shown in FIGS. 19A and 19B comprises a hose barb. The mechanism for attaching to the tube 1948 can comprise threads, barbs, CPC fittings, or any other suitable tubing connection mechanisms.

The coupler 1950 may comprise an attachment portion 1954 configured to securely attach the coupler 1950 to a proximal end portion of a first shaft. For example, as shown in FIGS. 19A and 19B, the attachment portion 1954 may comprise female threads 1958 configured to receive male threads of 1940 the proximal end portion 1920b of the first shaft 1920. In some embodiments, the attachment portion 1954 comprises an elastomeric seal 1960 (e.g., an o-ring seal or flat seal) to improve the coupling between the coupler 1950 and the first shaft 1920. The proximal end portion 1920b of the first shaft 1920 may not be designed with threads or other attachment features, in which case the coupler 1950 might comprise a specialized clamp designed to engage the housing or handle of proximal end portion 1920b of the first shaft and hold coupler 1950 against it, preventing air and fluid leakage and inadvertent detachment for the period of mechanical support. The clamp might be designed to engage specific features of the proximal end portion 1920b of the first shaft 1920 to hold it securely while also being ergonomically acceptable to be in close proximity to the patient for the period of mechanical support. For example, the clamp might have rounded edges and minimal size and weight.

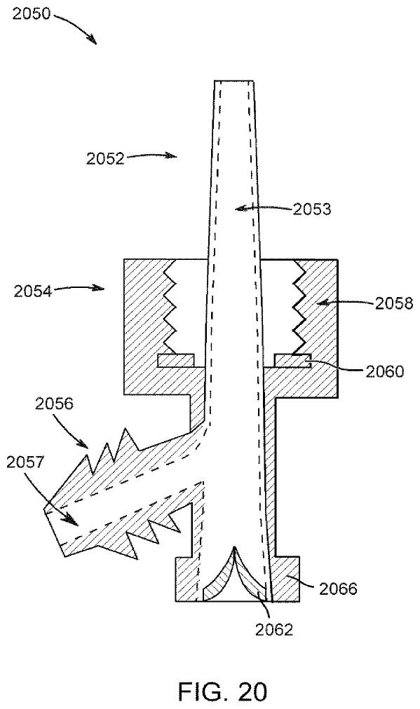

FIG. 20 is a cross-sectional view of a coupler 2050 comprising a shaft 2052, an attachment portion 2054, and an outflow channel 2056 in accordance with several aspects of the present technology. A lumen 2053 extends through the shaft 2052. The distal end portion of the lumen 2053 may be open to be configured to connect to a proximal end portion of a first elongated shaft. The proximal end portion of the lumen 2053 may be open to receive a delivery catheter through the lumen 2053. The coupler 2050 may comprise a hemostatic valve 2062 positioned within the lumen 2053 as shown in FIG. 20 to prevent blood leakage and/or air inflow. The hemostatic valve 2062 may comprise seals, flaps, plugs, caps, or other suitable features to prevent fluid or air passage through the hemostatic valve 2062.

In some embodiments, for example as shown in FIG. 20, the coupler 2050 may comprise an outflow channel 2056 positioned at an angle relative to a longitudinal axis of the coupler 2050. The distal end portion of the lumen 2057 of the outflow channel 2056 can be open to the lumen 2053 of the shaft and the proximal end portion of the lumen 2047 of the outflow channel 2056 can be open to a lumen of tubing leading to the pressure source and/or a second elongated shaft to fluidly connect the first elongated shaft with the other components of the system. The outflow channel 2056 may also be directly connected to the pressure source, and the pressure source may be a part of the coupler 2050. The outer surface of the outflow channel 2056 may comprise a hose barb or other suitable tubing connection mechanism as previously described. In some embodiments, for example as shown in FIG. 20, the outflow channel 2056 can be integrally formed with the shaft 2052 and/or attachment portion 2054 of the coupler 2050. The outflow channel 2056 may be detachably coupled to the shaft 2052 and/or attachment portion 2054. In some embodiments, the outflow channel 2056 rotates relative to the shaft 2052 and/or attachment portion 2054.

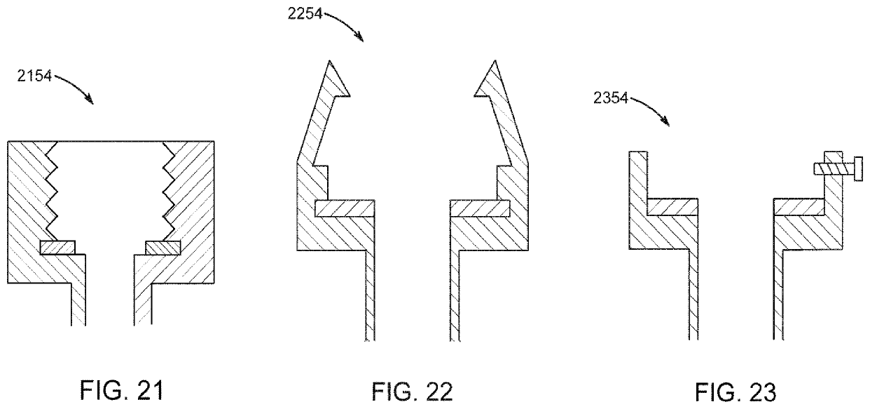

The attachment portion 2054 of the coupler 2050 is configured to securely and/or removably attach a first elongated shaft to the coupler 2050. As shown in FIG. 20 and previously described regarding FIGS. 19A and 19B, the attachment portion 2054 may comprise threads 2058 and/or an elastomeric ring 2060. FIGS. 21-23 depict various embodiments of attachment portions in accordance with the present technology. For example, a coupler may comprise an attachment portion 2154 with threads (see FIG. 21), an attachment portion 2254 comprising a snap-fit mechanism (see FIG. 22), an attachment portion 2354 comprising a set screw mechanism (see FIG. 23), or a clamp. In some embodiments, an attachment portion of the present technology does not comprise an elastomeric ring.

A connector in accordance with the present technology may comprise a coupler (as previously described) and/or a tube. In some embodiments, a coupler is attached to a proximal end portion of an elongated shaft (i.e., first or second elongated shaft) and a distal end portion of the tube is attached to an outflow channel of the coupler. In some embodiments, the distal end portion of the tube is directly attached to the proximal end portion of an elongated shaft. A proximal end portion of the tube may be attached to another tube, a pressure source, or another elongated shaft. For example, in some embodiments, a distal end portion of the tube attaches to an outflow channel of a coupler and a proximal end portion of the tube attaches to a pressure source. In some embodiments, the pressure source is directly coupled to or a part of the coupler.

The tube may comprise medical grade tubing formed of a suitable material such as polyvinyl chloride (PVC). The tube may have an inner diameter between about 0.250 inches to 0.5 inches. In some embodiments, the inner surface of the tube is coated with an anti-coagulant such as heparin or another suitable coating to minimize clotting, blood damage, and/or inflammatory response.

The tube can connect the coupler to the pressure source and the pressure source to the second elongated shaft. In some embodiments, for example when the pressure source comprises a roller pump, the tube may be inserted into the pressure source. The pressure source can comprise a centrifugal pump, a peristaltic pump, a pulsatile pump, roller pump, or any other pump suitable for moving blood. In some embodiments. the pump comprises an oxygenator to introduce oxygen into the blood before the blood is advanced out of the distal end region of the second shaft into a patient's artery. According to some embodiments, the pressure source is directly connected to or integral with the first elongated shaft, the coupler, the tube, and/or the second elongated shaft.

### IV. Selected Second Shaft Embodiments

According to some embodiments, a system of the present technology comprises a second elongated shaft configured to be positioned within an arterial vessel of the patient such that a distal end portion of the second shaft is positioned downstream of a distal end portion of a first shaft. In some embodiments, the second shaft is a return cannula. The second shaft can comprise an outer diameter between about 12 French and about 24 French, an inner diameter between about 10 French and about 22 French, and/or a length between about 8 cm and about 50 cm. The outer diameter, inner diameter, and/or length of the second shaft may be any suitable value based on the anatomy of the patient to be treated. The second shaft may be formed of a material such as a thermoplastic elastomer (e.g., Pebax®), polyurethane, or another material suitable for forming catheters or return cannulas. The second shaft may comprise a material such as, but not limited to, wire, a coil, or a braid, within a sidewall of the second shaft for reinforcement, and/or kink-resistance. The second shaft may comprise one or more steerable regions, as described elsewhere herein.

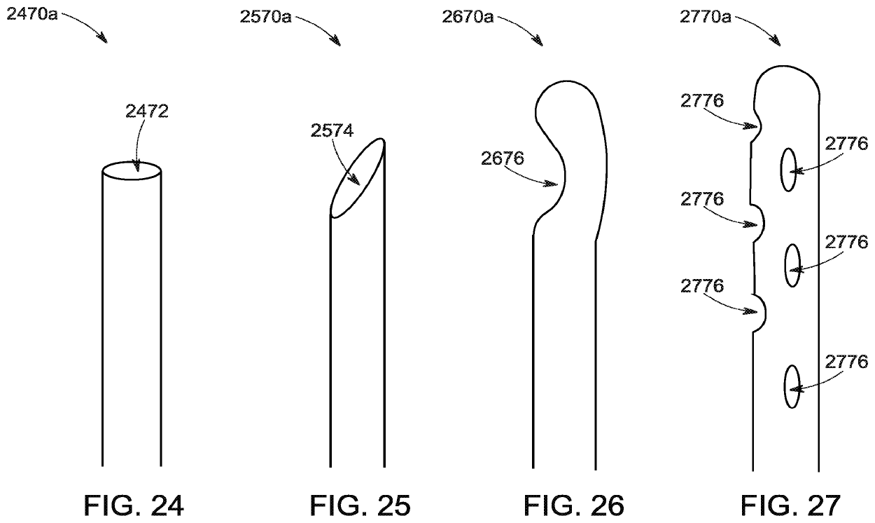

The second elongated shaft is configured to deliver blood to a patient's arterial circulatory system. Accordingly, the second elongated shaft comprises one or more openings for release of blood from the second shaft. FIGS. 24-27 illustrate various embodiments of such openings. A distal end portion 2470a of the second elongated shaft may comprise an open lumen 2474 having a generally blunt distal end as shown in FIG. 24. In some embodiments, the distal end portion 2570a comprises an open lumen 2574 but the distal end has a beveled shape, for example as shown in FIG. 25. According to some embodiments, the distal end portion 2670a of the second shaft comprises a closed and/or atraumatic distal terminus and a side hole 2676 extending through a sidewall of the second shaft (see FIG. 26). The distal end portion 2770a may comprise a plurality of side holes 2776 as shown in FIG. 27.

### V. Conclusion

Although many of the embodiments are described above with respect to systems and methods for mechanical circulatory support related to transcatheter heart valve repair or replacement, the present technology is applicable to other applications and/or other approaches, such as any transcatheter heart therapy. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-27.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

### VI. References

[1] A. P. S. Huang and R. K. Sakata, “Pain after sternotomy—review,” Brazilian Journal of Anesthesiology (English Edition), vol. 66, no. 4, pp. 395-401, July 2016.
[2] C. Heilmann et al., “Wound complications after median sternotomy: a single-centre study,” Interactive CardioVascular and Thoracic Surgery, vol. 16, no. 5, pp. 643-648, May 2013.
[3] M. J. Czarny and J. R. Resar, “Diagnosis and Management of Valvular Aortic Stenosis,” Clinical Medicine Insights: Cardiology, vol. 8s1, p. CMC. S15716, January 2014.
“Heart Disease and Stroke Statistics-2019 Update: A Report From the American Heart Association,” p.e456.
[5] M. J. Czarny and J. R. Resar, “Diagnosis and Management of Valvular Aortic Stenosis,” Clinical Medicine Insights: Cardiology, vol. 8s1, p. CMC.S15716, January 2014.
[6] M. B. Leon et al., “Transcatheter or Surgical Aortic-Valve Replacement in Intermediate-Risk Patients,” New England Journal of Medicine, vol. 374, no. 17, pp. 1609-1620, April 2016.
[7] D. H. Adams et al., “Transcatheter Aortic-Valve Replacement with a Self-Expanding Prosthesis,” New England Journal of Medicine, vol. 370, no. 19, pp. 1790-1798, May 2014.
[8] https://www-uptodate-com.laneproxy.stanford.edu/contents/clinical-manifestations-and-diagnosis-of-chronic-mitral-regurgitation? search=mitral %20valve %20regurgitation&source=search_result&selectedTitle=1˜150&usage_type=default&display_rank=1 accessed May 13, 2019.
[9] “Heart Disease and Stroke Statistics—2019 Update: A Report From the American Heart Association,” p.e458.
[10] U. Schäfer, F. Deuschl, N. Schofer, E. Lubos, and S. Blankenberg, “Critical evaluation of the MitraClip system in the management of mitral regurgitation,” Vascular Health and Risk Management, p. 1, January 2016.
[11] G. Nickenig et al., “Percutaneous Mitral Valve Edge-to-Edge Repair,” Journal of the American College of Cardiology, vol. 64, no. 9, pp. 875-884, September 2014.
[12] D. D. Glower et al., “Percutaneous Mitral Valve Repair for Mitral Regurgitation in High-Risk Patients,” Journal of the American College of Cardiology, vol. 64, no. 2, pp. 172-181, July 2014.
[13] Chandrashekhar Y, Westaby S, Narula J. Mitral stenosis. The Lancet 2009; 374:1271-83.doi:10.1016/S0140-6736(09)60994-6.
[14] https://www-uptodate-com.laneproxy.stanford.edu/contents/surgical-and-investigational-approaches-to-management-of-mitral-stenosis?search=mitral %20valve %20replacement&source=search_result&selectedTitle=2˜107& usage_type=default&display_rank=2 accessed May 13, 2019.
[15] V. Singh, A. Yarkoni, and W. W. O'Neill, “Emergent use of Impella CP™ during transcatheter aortic valve replacement: Transaortic access: Impella CP in Transaortic TAVR,” Catheterization and Cardiovascular Interventions, vol. 86, no. 1, pp. 160-163, July 2015.
[16] E. Dolmatova et al., “Extracorporeal membrane oxygenation in transcatheter aortic valve replacement,” Asian Cardiovascular and Thoracic Annals, vol. 25, no. 1, pp. 31-34, January 2017.
[17] S. N. Doshi, “Use of Left Ventricular Support Devices During TAVR,” p. 5.
[18] V. Singh, A. Yarkoni, and W. W. O'Neill, “Emergent use of Impella CP™ during transcatheter aortic valve replacement: Transaortic access: Impella CP in Transaortic TAVR,” Catheterization and Cardiovascular Interventions, vol. 86, no. 1, pp. 160-163, July 2015.

1. A system for providing cardiac support to a patient, the system comprising:
a first elongated shaft defining a first lumen extending therethrough, the first shaft having a proximal end portion and a distal end portion, wherein the distal end portion is configured to be intravascularly positioned at a first cardiovascular location, and wherein the lumen of the first shaft is configured to slidably receive a catheter housing an interventional element in a low-profile state;
a second elongated shaft defining a second lumen extending therethrough, the second shaft having a proximal end region and a distal end region, wherein the distal end region is configured to be intravascularly positioned at a second cardiovascular location within an artery of the patient; and
a pressure source configured to generate pressure within the first lumen and the second lumen, wherein the pressure source is configured to be coupled to the proximal end portion of the first shaft and the proximal end region of the second shaft, and wherein pressure generated by the pressure source pulls blood from the first location proximally through the first shaft to the pressure source, then pushes the blood distally through the second shaft and into circulatory flow at the second cardiovascular location, thereby providing mechanical circulatory support to the patient.

2. The system of claim 1, wherein the pressure source is configured to generate the blood flow while the catheter is positioned within and/or extending distally from the distal end portion of the first shaft.

3. The system of claim 1, wherein the pressure source is configured to be extracorporeally positioned while generating pressure.

4. The system of claim 1, further comprising an oxygenator configured to oxygenate the blood as it flows between the distal end portion of the first shaft and the distal end region of the second shaft.

5. The system of claim 1, wherein the first cardiovascular location is within one of the left ventricle, the left atrium, or the ascending aorta.

6. The system of claim 1, wherein the second cardiovascular location is within one of the ascending aorta, the aortic arch, the descending aorta, the subclavian artery, or the femoral artery.

7. The system of claim 1, wherein the distal end portion of the first shaft comprises a plurality of openings extending through a sidewall of the first shaft.

8. The system of claim 1, wherein a radial dimension of the distal end portion of the first shaft decreases in a distal direction.

9. The system of claim 1, wherein the first shaft comprises a plurality of projections extending radially inwardly from an inner surface of the first shaft.

10. The system of claim 9, wherein some or all of the projections comprise a curved surface that is convex toward the first lumen.

11. The system of claim, wherein the distal end portion of the first shaft is configured to be positioned across a septum.

12. The system of claim 1, wherein the interventional element comprises a prosthetic mitral valve.

13. The system of claim 1, wherein the interventional element comprises a prosthetic aortic valve.

14. The system of claim 1, wherein the interventional element comprises a heart valve repair device.

15. A system comprising:
a bypass device comprising a first end region with an inlet, a second end region with an outlet, and a fluid path extending therebetween, wherein the first end region is configured to be intravascularly delivered to and positioned at a first cardiovascular location, and wherein the second end region is configured to be intravascularly delivered to and positioned at a second cardiovascular location within an artery of the patient; and
a pressure source disposed along the fluid path between the inlet and the outlet,
wherein a portion of the bypass device between the pressure source and the inlet is configured to receive a catheter containing an interventional element, and wherein, when the pressure source is activated, the pressure source pulls blood from the first cardiovascular location into the inlet, through the fluid path, and ejects the blood from the outlet to the second cardiovascular location.

16. The system of claim 15, wherein the pressure source is configured to aspirate blood from the first location and eject blood to the second location while the catheter is positioned within the bypass device.

17. The system of claim 15, wherein the pressure source is a pump.

18. The system of claim 17, wherein the pump is a centrifugal pump, a peristaltic pump, a pulsatile pump, or a roller pump.

19. The system of claim 15, wherein the interventional element comprises a heart valve repair device.

20. A system for providing cardiac support to a patient, the system comprising:
an inlet catheter defining a first lumen extending therethrough, the inlet catheter having a proximal end portion and a distal end portion, wherein the distal end portion is configured to be intravascularly positioned at a first arterial location, and wherein the lumen of the inlet catheter is configured to slidably receive a delivery catheter housing a prosthetic heart valve in a low-profile state;
an outlet catheter defining a second lumen extending therethrough, the outlet catheter having a proximal end region and a distal end region, wherein the distal end region is configured to be intravascularly positioned at a second arterial location; and
a pump configured to be coupled to the proximal end portion of the inlet catheter and the proximal end region of the outlet catheter, and wherein pressure generated by the pump pulls blood from the first arterial location proximally through the inlet catheter to the pump, then pushes the blood distally through the outlet catheter and into circulatory flow at the second arterial location, thereby providing mechanical circulatory support to the patient.
